(19) United States
(12) Patent Application Publication
(10) Pub. No.: US 2004/0048238 A1
(43) Pub. Date: Mar. 11, 2004

(54) Method and assay for diagnosing substance dependency

(76) Inventors: Terence C. Town, Tampa, FL (US); Laila Abdullah, Tampa, FL (US); Michael Mullan, Tampa, FL (US); John Andrew Schinka, Lutz, FL (US)

Correspondence Address: Barbara E. Johnson, Webb Ziesenheim Logsdon Orkin & Hanson, P.C., 700 Koppers Building, 436 Seventh Avenue, Pittsburgh, PA, 15219-1818, US

(21) Appl. No.: 10/638,571

(22) Filed: Aug. 11, 2003

Related U.S. Application Data
(62) Division of application No. 09/898,566, filed on Jul. 2, 2001, now abandoned.
(60) Provisional application No. 60/215,506, filed on Jun. 30, 2000.

(51) Int. Cl. C12Q 1/00

(52) U.S. Cl. 435/4

(57) ABSTRACT

There is provided an assay for assessing the risk for developing substance dependency, including alcohol, opiate, cocaine, marijuana having a screening device for screening samples for the OPRM1+118A allele polymorphism, the presence of the allele indicating a risk of developing substance dependency. A method for diagnosing the risk for developing substance dependency by screening samples for the OPRM1+118A allele, whereby the presence of the allele indicates substance dependence is also provided. Also provided is a marker for determining the risk of developing substance dependency having an allele indicating the risk for developing substance dependency.

### CROSS REFERENCE TO RELATED APPLICATIONS

This application claims the benefit of priority under 35 U.S.C. Section 119(e) of U.S. Provisional Patent Application No. 60/215,506, filed Jun. 30, 2001 which is incorporated herein by reference.

### TECHNICAL FIELD

The present invention relates to methods for diagnosing alcohol dependency and other substance abuse. More specifically, the present invention relates to methods of both assessing an individual's risk for developing dependency on alcohol and other addictive substances and supplementing current diagnostic criteria for alcohol and other substance dependency by genotyping human subjects, to detect the presense or absense of the +118A/G m-opioid receptor allele polymorphism.

### BACKGROUND ART

Alcoholism is a clinically heterogeneous disorder of moderate heritability with a pattern of genetic transmission not readily explained by single gene models (Merikangas, 1990; Kendler et al., 1992). Indeed, the contribution of environmental factors and associated co-morbid psychological conditions to alcoholism vulnerability complicates elucidation of disease etiology.

For example, the Taql A1 allele of the dopamine receptor D2 (DRD2) gene has been associated with alcohol dependency (Blum et al., 1993; Arinami et al., 1993; Ishiguro et al., 1998). However, the association has not been consistently replicated (Suarez et al., 1994; Lu et al., 1996; Lawford et al., 1997). One reason for the inconsistency may be due to the variation in sampling methodologies; for example, ethnic, regional, and gender differences between studies.

The DRD2 Taql A1 association with alcoholism results reveal that the percentage of disease variance explained by this allele is small.

The endogenous m-opioid system is thought to be involved in the etiology of alcohol dependency in humans. For instance, patients carrying the +118A allele polymorphism of the m-opioid gene (OPRM1) demonstrate enhanced sensitivity to alcohol withdrawal as measured by apomorphine-induced growth hormone (a marker of dopaminergic sensitivity) secretion (Smolka et al., 1999). Another polymorphism within the m-opioid gene, known as a non-coding, non-functional (CA)n repeat polymorphism, has been associated with polysubstance abuse such as alcohol dependency (Kranzler et al., 1998). The functional coding polymorphism +118A/G within OPRM1 which codes for the receptor isoform has been shown to differentially affect receptor affinity for b-endorphin (the endogenous ligand for the m-opioid receptor) (Bond et al., 1998), the receptor isoform predicted by the +118A allele binds endogenous b-endorphin approximately 3 times less tightly than the +118G receptor isoform. Consequently alcoholics exhibit diminished b-endorphin blood plasma levels and decreased hypothalamic-opioid activity than unaffected controls (Inder et al., 1998; Wand et al., 1998), may suggest that specific alleles of the m-opioid receptor gene may be risk factors for alcoholism. For example, the Taql A1 allele of the dopamine receptor D2 (DRD2) gene has been associated with alcohol dependency (Blum et al., 1993; Arinami et al., 1993; Ishiguro et al., 1998). However, the association has not been consistently replicated (Suarez et al., 1994; Lu et al., 1996; Lawford et al., 1997), as would be expected for a disease of polygenic inheritance. Thus, researchers have continued to search for other genetic loci that may, either alone or in combination with DRD2, enhance prediction of alcoholism.

The +118A/G polymorphism within OPRM1 has been suggested to be a risk factor for other substance dependency. Twin studies not only reveal large (i.e., ≧50%) genetic components to risk for disorders such as alcohol dependence⁴ and cigarette smoking (True et al., 1997), but have also shown that a substantial portion of the genetic risk for substance dependence disorders is shared in common. A recent study employing the Vietnam Era Twin Registry, reported that 25.5% of the genetic risk for DSM-IIIR diagnosed alcohol and nicotine dependence was common to both disorders (True et al., 1999). However, the Sander et al. reference concludes that “Our results do not provide evidence that the common Asn40Asp substitution polymorphism of the OPRM gene contributes a major effect to the pathogenesis of alcohol dependence.”

It would therefore be useful to identify a genetic loci that predicts, alone or in association with other genetic loci the risk of alcoholism and other substance dependence. Further, the identification of an allele variance or polymorphism that is consistently found in association with this indication would be useful, particularly in view of the polygenic nature of the disorder. It is a further object of the invention to utilize the +118A/G polymorphism within OPRM1 to predict risk for alcoholism and other substance dependence.

### SUMMARY OF THE INVENTION

According to the present invention, there is provided an assay for determining the tendency for alcohol dependency by an individual and supplementing current diagnostic strategies for treating alcoholism by screening samples for OPRMI+118A allele polymorphism. The method includes screening samples containing genetic material such as body fluids including peripheral blood for the presence of the OPRM1+118A allele polymorphism by genotyping methods, such as using +118A/G OPRM1 restriction fragment length polymorphism. The presence of the OPRM1+118A allele polymorphism in such sample correlates with alcohol dependency. Additionally, a method is provided for genotyping the OPRM1+118A allele polymorphism as a predictor for other substance abuse. Also provided is a marker for determining the risk of developing substance dependency comprising an allele polymorphism indicating the risk for developing substance dependency.

### BRIEF DESCRIPTION OF THE FIGURES

Other advantages of the present invention will be readily appreciated as the same becomes better understood by reference to the following detailed description when considered I connection with the accompanying drawings wherein:

FIG. 1 is a table showing DRD2 Taql polymorphism frequencies in alcoholics (n=105) and controls (n=210), no association was detected between DRD2 Taql polymorphism and alcoholism by allele or genotype;

FIG. 2 is a table demonstrating OPRM1+118A/G polymorphism frequencies in alcoholics (n=105) and controls (n=210), there is a significant association between OPRM1+118A/G polymorphism and alcoholism by both genotype (p=0.020) and allele (p=0.019).

FIG. 3 is a table showing the genotypes, alleles, and frequencies of OPRMI+118A/G.

### DETAILED DESCRIPTION OF THE INVENTION

Generally, the present invention provides an assay for assessing an individual's risk for developing alcohol dependency and other substance dependence (including smoking, opiate abuse). The assay functions by screening samples, such as peripheral blood, for the presence of the OPRM1+118A allele polymorphism. In addition, the invention provides supplement diagnostic strategies for treating alcoholism and other addictive substances by screening samples, such as peripheral blood, for the presence of the OPRM1+118A allele polymorphism.

Alcohol dependency, alcoholism, is a primary, chronic disease with genetic, psychosocial and environmental factors influencing its development and manifestations. The disease is often progressive and fatal. It is characterized by continuous or periodic: impaired control over drinking, preoccupation with the drug alcohol, use of alcohol despite adverse consequences and distortions in thinking, most notably denial. The dependency on other addictive substances is similar to the alcoholism and therefore applicants determined that an assay for alcoholism would also function for diagnosing a predisposition to other substance dependency. This screening method permits screening of individuals for predisposition for alcoholism by withdrawing a genetic sample, for example, peripheral blood, then performing genotyping procedures to detect the the presence of the OPRM1+118A allele polymorphism.

Samples containing DNA such as peripheral blood are tested for the presence of OPRM1+118A allele polymorphism. For example, peripheral blood samples are prepared using standard phenol-chloroform methods or the Puregene™ kit (Gentra Systems). Other methods known to those of skill in the art can also be used for detecting the presence of the allele. These methods include methods for detecting phenotypic changes, which methods include but are not limited to methods of detecting the amino acid change in the expressed protein, antibody detection by immunoprecipitation, Western blotting, or other phenotyping methods known to those of skill in the art.

Genotyping of the samples is preferably performed using the +118A/G OPRMI restriction fragment length polymorphism (RFLP) amplified using primers and conditions described by Bergen et al. (1997). Other methods of genotyping as are known to those of skill in the art can also be used in accordance with the mehtods of the present invention these can include for example allele specific polymerase chain reaction (Sander et al.). The amplified fragment is 95 bp and an AG substitution (predicting an Asn-to-Asp amino acid substitution at position 40 of the receptor) creats a Drdl recognition sequence, which when digested results in 22 bp and 73 bp fragments.

Also provided by the present invention is a marker for determining the risk of developing substance dependency. The marker is preferably OPRM1+118A allele polymorphism. The marker functions such that the presence of the marker, that is the AA genotype and A allele in a tissue sample serves to indicate an increased risk of developing or having a substance dependency. The substance dependency can be alcohol dependency or dependency on other addictive substances.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE

### Materials and Methods

Study subjects

The alcohol dependent sample consisted of individuals recruited from the James A. Haley Veterans Hospital in Tampa, Fla. Each individual met the criteria for alcohol dependency as defined by DSMIV. The mean age of the alcohol dependent sample was 47.1±10.3 years, primarily Caucasian (81.9%), composed of 104 males (99.04%) and 1 female.

The control sample consisted of 122 individuals, selected as part of a larger epidemiologic study based in the Miami area of Florida. The mean age of the control sample was 73±9.44 years, primarily Caucasian (78.7%), composed of 73 males (34.7%) and 137 females (65.3%). All members of the control sample were over 50 years of age, allowing for sufficient exposure to risk for alcohol abuse. Indeed, individuals in the control sample consumed no more than one alcoholic drink per week and had no prior history of behaviors linked to DRD2 Taql A1 allele such as drinking, smoking, polysubstance abuse, or family history of addictive behavior.

Another example was also employed, consisting of 179 Caucasians who were unrelated individuals recruited from the substance abuse treatment program of a large, metropolitan VA medical center. All cases met the criteria for alcohol dependency as defined by DSM IV and reported alcohol as the primary drug of dependency. Data from 80 of these cases were reported in the previous study.⁷ Detailed information about history of substance use was obtained by means of a structured interview administered by trained graduate-level research assistants. The mean age of the alcoholic sample was 46.9±8.6 years and all but 4 cases were males. Three subsamples were also selected from this patient group on the basis of structured interviews of substance abuse history. These included: a) the ETOH-NIC-DRUG group, 43 cases with a history of abuse of other substances (alcohol, nicotine and primarily cocaine and marijuana) and current daily use of cigarettes (27.9±10.1 cigarettes per day), b) the ETOH-NIC group, 91 cases with no history of abuse of other illegal substances who were current smokers (30.3±14.8 cigarettes per day), and c) ETOH ONLY group, 17 cases with no history of use of illegal substances or daily use of cigarettes.

This control sample consisted of 297 Caucasians (48.1% male) who were selected as part of a larger epidemiologic study of healthy elderly residing on the west coast of Florida¹² and had not been participants in any of the previous studies. Information regarding history and current use of alcohol and cigarettes was obtained as part of an extensive structured evaluation conducted by trained interviewers, as described in detail by Small et al.¹² All cases in this sample were over the age of 60 (mean age=72.8±6.1), thus guaranteeing exposure to risk for alcohol abuse and nicotine dependence. The sample was characterized by individuals with varying histories of use of alcohol and cigarettes. A group of 63 individuals, all of whom had a lifetime history of less than one alcoholic drink per month and less than one cigarette per week, was selected from the larger unrestricted control sample to serve as a super control sample.

Genotyping

DNA was extracted from peripheral sample with Puregene™ kit (Gentra Systems) or by using standard phenol-chloroform methods. The +118A/G OPRMI restriction fragment length polymorphism (RFLP) was amplified using primers and conditions previously described by Bergen and colleagues (1997). The amplified fragment is 95 bp, and an A-G substitution (predicting an Asn-to-Asp amino acid substitution at position 40 of the receptor) creates a Drdl recognition sequence, which when digested, results in 22 bp and 73 bp fragments.

Genotyping for the DRD2 Taql RFLP was performed using primers and conditions described by Grandy and colleagues (1989). The amplified fragment is 310 bp (corresponding to the A1 allele). Complete digestion results in 180 bp and 130 bp fragments (corresponding to the A2 allele).

### Statistical Analysis

The likelihood ratio χ² statistic was used to compare genotype and allelic distributions between control and alcoholic samples as well as between genders within the control group. Multiple logistic regression models were used to assess retrospective ORs for each genotype or allele, to assess interaction between the OPRM1 and DRD2 polymorphisms on prediction of alcoholism, and to determine the effect of ethnicity on prediction of alcohol dependency.

ORs and corresponding 95% confidence intervals (Cis) were calculated according to standard methods. Alpha levels were set at 0.05 for each analysis, which was performed using SPSS for windows release 7.5.1. Power analysis was performed using Power and Precision release 1.0.

### Results

Association Between DRD2 Taql Polymorphism and Alcoholism and Between OPRM1+118A/G Polymorphism and Alcoholism by Genotype or Allele.

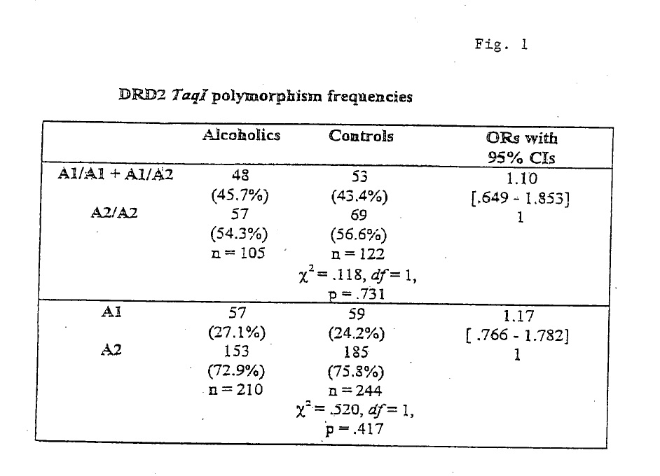

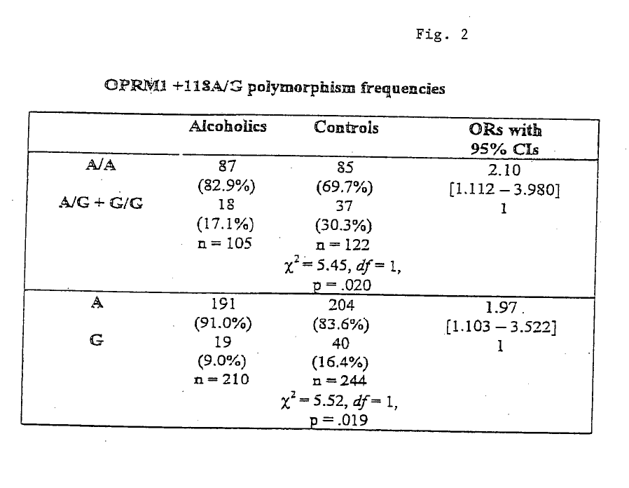

No association is detected between the DRD2 Taql polymorphism and alcoholism, either by genotype or allele (FIG. 1). However, unexpectedly, there is a significant association between OPRM1+118A/G polymorphism and alcoholism, both by genotype (p=0.020) and allele (p=0.019, FIG. 2). Indeed, Odds Ratios (Ods) show that +118A homozygotes carry a greater than 2-fold risk for alcoholism. Further the +118A allele polymorphism confers approximately a 2-fold risk for alcoholism.

Logistic regression models including an interactive term (DRD2*OPRM1) showed that there was no significant synergistic effect on prediction of alcoholism by either allele (p=0.103) or genotype (p=0.103) for DRD2 Taql and OPRM1+118 A/G polymorphisms. Thus, there was no synergistic effect related to the risk for the development of alcoholism.

Association Between Gender and DRD2 or OPRM1 Polymorphism for the Risk of Alcoholism

There are no significant differences between DRD2 Taql polymorphism status in males and females by allele or genotype. For example by allele, the A1 frequency in females was 25.9% as compared to 21.4% in males (p=0.432). By genotype, A1/A1+A2/A2 frequency was 46.8% in females as compared to 38.1% in males (p=0.355).

Furthermore, OPRM1+118 A/G status did not significantly differ between genders by allele or genotype. By allele, the A frequency was 86.1% in females as compared to 78.6% in males (p=0.140). Similarly, by genotype, the A/A frequency was 73.4% in females and 61.9% in males (p=0.194). The x-square statistic showed the OPRM1 genotype distributions in case (P=0.56) and control (P=0.69) samples to be in Hardy-Weinberg equilibrium. There was no relationship between gener and OPRM1 genotype in the full control sample (P=0.36).

Association Between Ethnicity and DRD2 or OPRM1 Polymorphism for the Risk of Alcoholism.

Analysis performed to determine the effects of ethnicity on prediction of alcohol dependency show that there is no interaction between ethnicity and either DRD2 Taql or OPRM1+118A/G by allele or genotype. Furthermore, logistic regression models did not show a major effect of ethnicity on prediction of alcoholism (p=0.556).

Association Between OPRM1 Polymorphism and Risk of Substance Abuse.

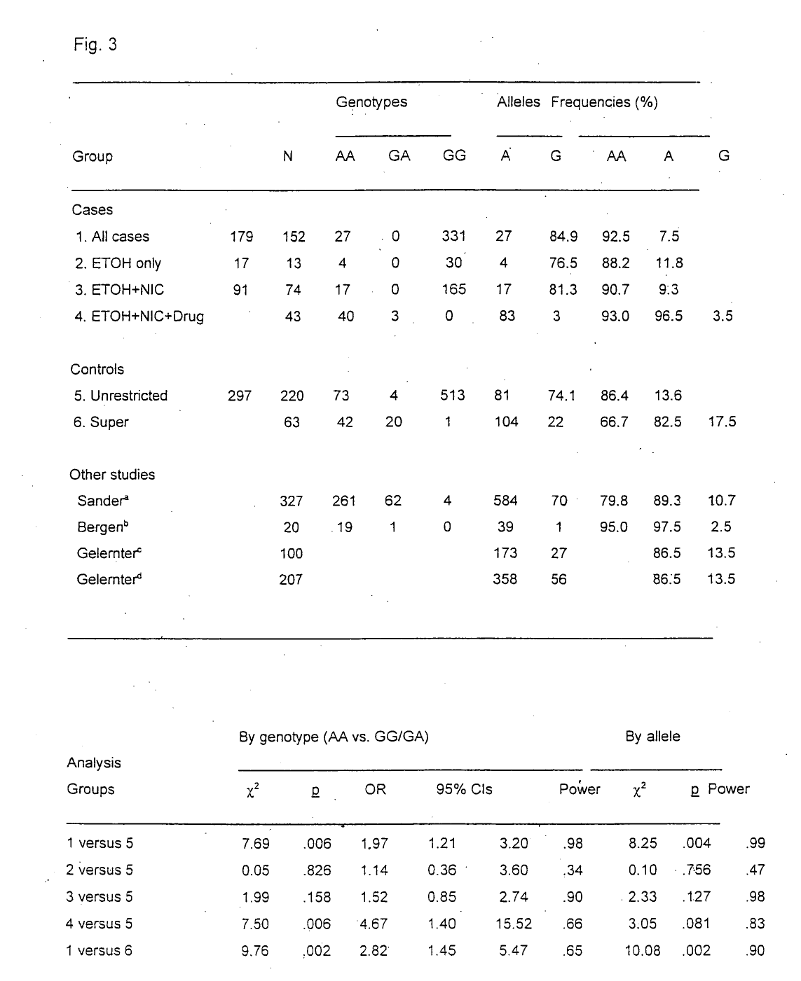

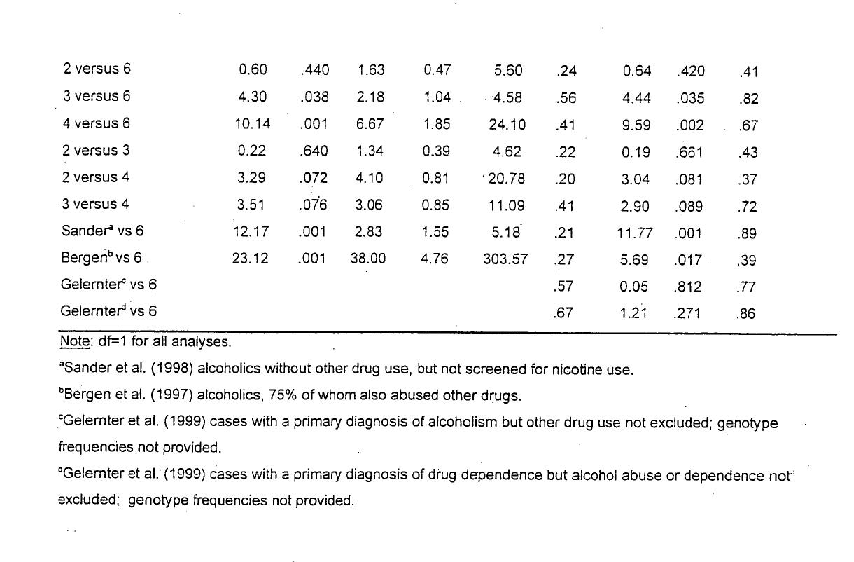

FIG. 3 presents genotype and allele frequencies for case and control samples and subsamples and the results of χ² analyses for frequency comparisons of the AA versus AG/GG genotypes and the A and G alleles among samples. Inspection of the table reveals an increasing frequency of the AA genotype and A allele corresponding with an increase in the number of drugs used among the alcoholic subsamples (ETOH+NIC+DRUG>ETOH+NIC >ETOH ONLY). Super controls have a lower frequency of the M genotype (66.7% versus 74.1%) and the A allele (82.5% versus 86.4%) than the unrestricted controls.

Using the unrestricted control sample as a reference group, the entire group of alcoholics was found to have a significantly higher frequency of the AA genotype (c²=7.69, p=0.006) and the A allele (C²=8.25, p=0.004), with approximately a twofold increase in risk for cases with the AA genotype. Analysis of alcoholic subsamples, however, revealed that only the ETOH+NIC+DRUG group had a significantly higher frequency of the AA genotype (C²=7.50, p=0.006). No case subsample had a significantly higher A allele frequency than unscreened controls.

A more revealing pattern of results, however, appears when using the super controls as a reference group. In analyses of genotypes, the entire group of alcoholics as well as all but the ETOH ONLY subsample have significantly higher frequencies of the AA genotype (ps<0.04), with ORs ranging from 2.18 to 6.67 (see FIG. 3). Although the ETOH ONLY group had approximately a 10% increase in AA genotype frequency in comparison to super controls, significant results were not found, possibly because of especially low power for the analysis (0.24). Parallel results were obtained for the analyses of the allele frequencies.

Examination of the genotype and allele frequencies among the alcoholic subsamples reveals no significant findings, although power for these analyses is low, exceeding 0.70 in only one instance. There is a trend (p<0.10) for the ETOH+NIC+DRUG group to have higher frequencies of the AA genotype and A allele than the other alcoholic subsamples.

FIG. 3 also presents genotype and allele frequencies for alcoholics and drug-dependent cases derived from tables presented in three previous studies examining the OPRM1+118 polymorphism in Caucasians, each of which failed to confirm a relationship between the polymorphism and alcoholism. Notably, two of these studies provide case genotype and allele frequencies similar to those of the alcoholic subsamples. The alcoholics without other illegal drug use described in the Sander et al. (1998) study have a similar frequency of the AA genotype (79.8%) and A allele (89.3%) as do the ETOH ONLY and ETOH+NIC subsamples. The alcoholics described by Bergen et al. (1997) were also largely (75%) illegal drug users and have a frequency of the AA genotype (95.0%) and A allele (97.5%) similar to the ETOH+NIC+DRUG subsample. The A allele frequencies reported by Gelernter et al. (1999) for separate samples of primary alcoholics and primary drug-dependent cases, however, are lower than those in any of the alcoholic subsamples.

Using the super control group as a reference group, the case samples of both the Sander et al. and the Bergen et al. studies were found to have a significantly higher frequency of the M genotype (c²=12.17, p=0.001 and c²=23.12, p=0.001, respectively). Analyses of the A allele frequencies yielded parallel findings (ps<0.02). Analyses for the Gelernter et al. samples, however, produced insignificant findings, despite adequate power.

For complex polygenic disorders the frequency of a specific risk allele may increase in groups with greater severity of the disorder. Thus, even mild use of alcohol or use of other addicitive substances such as nicotine may inflate risk allele rates in control samples and serve to minimize frequency differences between cases and controls. In support of this possibility, applicants obtained significant findings in analyses using both unscreened and super control groups, but with Ors that were approximately 50% higher when using super controls as the reference group for statistical analyses.

There is a consistent and orderly trend for higher frequencies of the AA geneotype and A allele in groups with increasing numers of substances used. Using the super control group as a referenc, Ors increase from 1.63 for alcoholics with no history of daily smoking or illegal substance abuse to 6.67 for those with a history of both.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

Arinami T, Itokawa M, Komiyama T, Mitsushio H, Mori H, Mifune H, Hamaguchi H, Toru M (1993): Association between severity of alcoholism and the Al allele of the dopamine D2 receptor gene Taql A RFLP in Japanese. Biol Psychiatry 33:108-114.

Bergen A W, Kokoszka J, Peterson R, Long J C, Virkkunen M, Linnoila M, Goldman D (1997): Mu-opioid receptor gene variants: lack of association with alcohol dependence. Mol Psychiatry 2:490-494.

Blum K, Noble E P, Sheridan P J, Finley O, Montgomery A, Ritchie T, Ozkaragoz T, Fitch R J, Sadiack F, Sheffield D, Dahimann T, Halbardier S, Nogami H (1991): Association of the Al allele of the D2 dopamine receptor gene with severe alcoholism. Alcohol 8:409-416.

Bond C, LaForge K S, Tian M, Melia D, Zhang S, Borg L, Gong J, Schluger J, Strong J A, Leal S M, Tischfield J A, Kreek M J, Yu L (1998): Single-nucleotide polymorphism in the human μ-opioid receptor gene alters beta-endorphin binding and activity: possible implications for opiate addiction. Proc Natl Acad Sci 95:9608-9613.

Comings D E, Ferry L, Bradshaw-Robinson S, Burchette R, Chiu C, Muhleman D (1996): The dopamine D2 receptor (DRD2) gene: A genetic risk factor in smoking. Pharmacogenetics 6:73-9.

Gelernter J, Kranzler H, Cubells J. Genetics of two μ opioid receptor gene (OPRM1) exon I polymorphisms: population studies, and allele frequencies in alcohol-and drug-dependent subjects. Mol Psych 1999; 4: 476-483.

Grandy D K, Litt M, Allen L, Bunzow M Marchionni M, Makam H, Reed L, Magenis R E, Civelli 0 (1989): The human dopamine D2 receptor gene is located on chromosome 11 at q22-q23 and identifies a Taql RFLP. Am J Hum Genet 45:778-785.

-fnder W J, Livesey J K Donald R A (1998): Peripheral plasma levels of beta-endorphin in alcoholics and highly trained athletes and the relationship to a measure of central opioid tone. Horm Metab Res 30:523-525.

Ishiguro R Arinami T, Saito T, Akazawa S, Enomoto M, Mitushio H, Fujishiro FL Tada K, AkimotoY, Mifune H, Shioduka S, Hamaguchi H, Toru M, Shibuya H (1998): Association study between the −141C Ins/Del and Taql A polymorphisms of the dopamine D2 receptor gene and alcoholism. Alcohol Clin Exp Res 22:845-848.

Kendier K S, Heath A C, Neale M C, Kessler R C, Eaves L J (1992): A population-based twin study of alcoholism in women. JAMA 268:1877-1882.

Kranzler H R, Gelernter J, OKrMaley S, Hernandez-Avila Calif., Kaufman D (1998): Association of alcohol or other drug dependence with alleles of the mMa-opioid receptor gene (OPRM1). Alcohol Clin Exp Res 22:1359-62.

Lawford B R, Young R N, Rowell J A, Gibson J N, Feeney G F, Ritchie T L, Syndulko K, Noble E P (1997): Association of the D2 dopamine receptor Al allele with alcoholism: medical severity of alcoholism and type of controls. Biol Psychiatry 41:386-393.

Lu R B, Ko H C, Chang F N L Castiglione C K Schoolfield G, Pakstis A J, Kidd M Kidd K K (1996): No association between alcoholism and multiple polymorphisms at the dopamine D2 receptor gene (DRD2) in three distinct Taiwanese populations. Biol Psychiatry 39:419-429.

Merikangas K R (1990): The genetic epidemiology of alcoholism. Psychol Med 20:11-22. Noble E P, Blum K (1993): Alcoholism and the D2 dopamine receptor gene. JAMA 270: 1547-8.

Sander T, Harms I L Dufeu P, Kuhn S, Rommelspacher R Schmidt L G (1997): Dopamine D4 receptor exon III alleles and variation of novelty seeking in alcoholics. Am J Med Genet 74:483-487.

Sander T, Gsheidel N, Wendel B, Samochowiec J, Smolka M, Rommelspacher H, Schmidt L G, Hoehe M R. Human μ-opioid receptor variation and alcohol dependence. AIc: Clin Exp Res 1998; 22: 2108-2110.

Self D W, Stein L (1992): Receptor subtypes in opioid and stimulant reward. Pharmacol Toxicol 70:87-94.

Smith SS, O'Hara B F, Perisco A M, Gorelick D A, Newlin D B, Vlahov D, Solomon L, Pickens R, Uhl G R (I 992): Genetic vulnerability to drug abuse: The D2 receptor Taql B restriction fragment length polymorphism appears more frequently in polysubstance abusers. Arch Gen Psychiatry 49:723-727.

Smolka M, Sander T, Schmidt L G, Samochowiec J, Rommelspacher H, Gscheidel N, Wendel B, Hoehe M R (1999): meh-opioid receptor variants and dopaminergic sensitivity in alcohol withdrawal. Psychoneuroendocrinology 24:629-38.

Suarez B K, Parsian A, Hampe C L, Todd R D, Reich T, Cloninger C R (1994): Linkage disequilibria at the D2 dopamine receptor locus (DRD2) in alcoholics and controls. Genomics 19:12-20.

True R W, Heath A C, Scherrer J F, Waterman B, Goldberg J, Lin N, Eisen S A, Lyons M J, Tsuang M T Genetic and environmental contributions to smoking. Addict 1997; 92, 1277-1287.

True R W, Xian H, Scherrer J F, Madden P A F, Bucholz K K, Heath A C, Eisen S A, Lyons M J, Goldberg J, Tsuang M T. Common genetic vulnerability for nicotine and alcohol dependence in men. Arch Gen Psych 1999; 56, 655-661.

Wand, G S, Mangold, D, El Deiry, S, McCaul, N I E, Hoover, D (1998): Family history of alcoholism and hypothalamic opioidergic activity. Arch Gen Psychiatry 55:1114-1119.

What is claimed is:

1. An assay for assessing the risk for developing or having substance dependency comprising screening means for screening samples for the OPRM1+118A allele, the presence of the allele indicating a risk of developing substance dependency.

2. The assay of claim 1, wherein screening means is genotyping means.

3. The assay of claim 2, wherein said screening means is phenotyping means.

4. The assay of claim 2, wherein samples include tissues sample including peripheral blood.

5. The assay of claim 1, wherein substance is alcohol.

6. The assay of claim 1 wherein substance is nicotine.

7. The assay of claim 1 wherein substance is cocaine.

8. The assay of claim 1 wherein substance is marijuana.

9. A method for estimating substance dependency comprising screening samples for the OPRM1+118A allele, the presence of the allele indicating an increased risk for substance dependence.

10. The method of claim 9, wherein screening step includes genotyping using +118A/G OPRM1 restriction fragment length polymorphism.

11. The method of claim 10, wherein said screening step includes screening tissue samples.

12. The method of claim 10, wherein said screening step includes screening peripheral blood.

13. The method of claim 9, wherein said screening step screens for diagnosing a substance dependency wherein the substance is alcohol.

14. The method of claim 9, wherein said screening step screens for diagnosing a substance dependency wherein the substance is nicotine.

15. The method of claim 9, wherein said screening step screens for diagnosing a substance dependency wherein the substance is cocaine.

16. The method of claim 9, wherein said screening step screens for diagnosing a substance dependency wherein the substance is marijuana.

17. A marker for determining the risk of developing substance dependency comprising the OPRM1+118A allele indicating the risk for developing substance dependency.

18. The marker of claim 17, wherein the substance is alcohol.

19. The marker of claim 17, wherein the substance is cocaine.

20. The marker of claim 17, wherein the substance is marijuana.
